SEQUENCE LISTING (78 sequences)

SEQ ID NO: 1 - protein, 17 aa, Artificial Sequence
  Synthetic
ATATATATATATATATA

SEQ ID NO: 2 - protein, 14 aa, Artificial Sequence
  Synthetic
VTVTVTVTVTVTVT

SEQ ID NO: 3 - protein, 12 aa, Artificial Sequence
  Synthetic
VTVTVTVTVTVT

SEQ ID NO: 4 - protein, 13 aa, Artificial Sequence
  Synthetic
TITITITITITIT

SEQ ID NO: 5 - protein, 11 aa, Artificial Sequence
  Synthetic
TITITITITIT

SEQ ID NO: 6 - protein, 11 aa, Artificial Sequence
  Synthetic
TFTFTFTFTFT

SEQ ID NO: 7 - protein, 9 aa, Artificial Sequence
  Synthetic
TFTFTFTFT

SEQ ID NO: 8 - protein, 17 aa, Artificial Sequence
  Synthetic
ATATATATATATATATA

SEQ ID NO: 9 - protein, 14 aa, Artificial Sequence
  Synthetic
VTVTVTVTVTVTVT

SEQ ID NO: 10 - protein, 12 aa, Artificial Sequence
  Synthetic
VTVTVTVTVTVT

SEQ ID NO: 11 - protein, 13 aa, Artificial Sequence
  Synthetic
TITITITITITIT

SEQ ID NO: 12 - protein, 11 aa, Artificial Sequence
  Synthetic
TITITITITIT

SEQ ID NO: 13 - protein, 11 aa, Artificial Sequence
  Synthetic
TFTFTFTFTFT

SEQ ID NO: 14 - protein, 9 aa, Artificial Sequence
  Synthetic
TFTFTFTFT

SEQ ID NO: 15 - protein, 12 aa, Unknown
  Mammalian
AASIKVAVSADR

SEQ ID NO: 16 - protein, 19 aa, Unknown
  Mammalian
CSRARKQAASIKVAVSADR

SEQ ID NO: 17 - protein, 5 aa, Unknown
  Mammalian
YIGSR

SEQ ID NO: 18 - protein, 5 aa, Unknown
  Mammalian
PDSGR

SEQ ID NO: 19 - protein, 7 aa, Unknown
  Mammalian
RYVVLPR

SEQ ID NO: 20 - protein, 12 aa, Unknown
  Mammalian
KAFDITYVRLKF

SEQ ID NO: 21 - protein, 12 aa, Unknown
  Mammalian
TAGSCLRKFSTM

SEQ ID NO: 22 - protein, 10 aa, Unknown
  Mammalian
RNIAEIIKDI

SEQ ID NO: 23 - protein, 4 aa, Unknown
  Mammalian
YVRL

SEQ ID NO: 24 - protein, 6 aa, Unknown
  Mammalian
IRVTLN

SEQ ID NO: 25 - protein, 8 aa, Unknown
  Mammalian
TTVKYIFR

SEQ ID NO: 26 - protein, 8 aa, Unknown
  Mammalian
SIKIRGTY

SEQ ID NO: 27 - protein, 13 aa, Unknown
  Mammalian
RQVFQVAYIIIKA

SEQ ID NO: 28 - protein, 10 aa, Unknown
  Mammalian
FQIAYVIVKA

SEQ ID NO: 29 - protein, 13 aa, Unknown
  Mammalian
GQLFHVAYIIIKA

SEQ ID NO: 30 - protein, 10 aa, Unknown
  Mammalian
FHVAYVLIKA

SEQ ID NO: 31 - protein, 13 aa, Unknown
  Mammalian
LENGEIVSLVNGR

SEQ ID NO: 32 - protein, 6 aa, Unknown
  Mammalian
LGTIPG

SEQ ID NO: 33 - protein, 4 aa, Unknown
  Mammalian
DGEA

SEQ ID NO: 34 - protein, 4 aa, Unknown
  Mammalian
REDV

SEQ ID NO: 35 - protein, 5 aa, Unknown
  Mammalian
GVGVP

SEQ ID NO: 36 - protein, 6 aa, Unknown
  Mammalian
GVGVAP

SEQ ID NO: 37 - protein, 5 aa, Unknown
  Mammalian
IKVAV

SEQ ID NO: 38 - protein, 7 aa, Unknown
  Mammalian
PFSSTKT

SEQ ID NO: 39 - protein, 8 aa, Unknown
  Mammalian
SKPPGTSS

SEQ ID NO: 40 - protein, 9 aa, Unknown
  Mammalian
SDPGYIGSR

SEQ ID NO: 41 - protein, 10 aa, Unknown
  Mammalian
RNIAELLKDI

SEQ ID NO: 42 - protein, 12 aa, Unknown
  Mammalian
PRGDSGYRGDSG

SEQ ID NO: 43 - protein, 7 aa, Unknown
  Mammalian
GFLGFPT

SEQ ID NO: 44 - protein, 7 aa, Unknown
  Mammalian
YGPDSGR

SEQ ID NO: 45 - protein, 24 aa, Artificial Sequence
  Synthetic
ATATATATATATATATAYGPDSGR

SEQ ID NO: 46 - protein, 23 aa, Artificial Sequence
  Synthetic
ATATATATATATATATAGIKVAV

SEQ ID NO: 47 - protein, 23 aa, Artificial Sequence
  Synthetic
ATATATATATATATATAGYIGSR

SEQ ID NO: 48 - protein, 25 aa, Artificial Sequence
  Synthetic
ATATATATATATATATAGPFSSTKT

SEQ ID NO: 49 - protein, 26 aa, Artificial Sequence
  Synthetic
ATATATATATATATATAGSKPPGTSS

SEQ ID NO: 50 - protein, 28 aa, Artificial Sequence
  Synthetic
ATATATATATATATATAGGSDPGYIGSR

SEQ ID NO: 51 - protein, 29 aa, Artificial Sequence
  Synthetic
ATATATATATATATATAGGRNIAELLKDI

SEQ ID NO: 52 - protein, 31 aa, Artificial Sequence
  Synthetic
ATATATATATATATATAGPRGDSDGYRGDSG

SEQ ID NO: 53 - protein, 22 aa, Artificial Sequence
  Synthetic
VTVTVTVTVTVTVTGYGPDSGR

SEQ ID NO: 54 - protein, 20 aa, Artificial Sequence
  Synthetic
VTVTVTVTVTVTVTGIKVAV

SEQ ID NO: 55 - protein, 20 aa, Artificial Sequence
  Synthetic
VTVTVTVTVTVTVTGYIGSR

SEQ ID NO: 56 - protein, 22 aa, Artificial Sequence
  Synthetic
VTVTVTVTVTVTVTGPFSSTKT

SEQ ID NO: 57 - protein, 23 aa, Artificial Sequence
  Synthetic
VTVTVTVTVTVTVTGSKPPGTSS

SEQ ID NO: 58 - protein, 25 aa, Artificial Sequence
  Synthetic
VTVTVTVTVTVTVTGGSDPGYIGSR

SEQ ID NO: 59 - protein, 26 aa, Artificial Sequence
  Synthetic
VTVTVTVTVTVTVTGGRNIAELLKDI

SEQ ID NO: 60 - protein, 27 aa, Artificial Sequence
  Synthetic
VTVTVTVTVTVTVTGPRGDSGYRGDSG

SEQ ID NO: 61 - protein, 21 aa, Artificial Sequence
  Synthetic
TITITITITITITGYGPDSGR

SEQ ID NO: 62 - protein, 19 aa, Artificial Sequence
  Synthetic
TITITITITITITGIKVAV

SEQ ID NO: 63 - protein, 19 aa, Artificial Sequence
  Synthetic
TITITITITITITGYIGSR

SEQ ID NO: 64 - protein, 21 aa, Artificial Sequence
  Synthetic
TITITITITITITGPFSSTKT

SEQ ID NO: 65 - protein, 22 aa, Artificial Sequence
  Synthetic
TITITITITITITGSKPPGTSS

SEQ ID NO: 66 - protein, 24 aa, Artificial Sequence
  Synthetic
TITITITITITITGGSDPGYIGSR

SEQ ID NO: 67 - protein, 25 aa, Artificial Sequence
  Synthetic
TITITITITITITGGRNIAELLKDI

SEQ ID NO: 68 - protein, 26 aa, Artificial Sequence
  Synthetic
TITITITITITITGPRGDSGYRGDSG

SEQ ID NO: 69 - protein, 19 aa, Artificial Sequence
  Synthetic
TFTFTFTFTFTGYGPDSGR

SEQ ID NO: 70 - protein, 17 aa, Artificial Sequence
  Synthetic
TFTFTFTFTFTGIKVAV

SEQ ID NO: 71 - protein, 17 aa, Artificial Sequence
  Synthetic
TFTFTFTFTFTGYIGSR

SEQ ID NO: 72 - protein, 19 aa, Artificial Sequence
  Synthetic
TFTFTFTFTFTGPFSSTKT

SEQ ID NO: 73 - protein, 20 aa, Artificial Sequence
  Synthetic
TFTFTFTFTFTGSKPPGTSS

SEQ ID NO: 74 - protein, 22 aa, Artificial Sequence
  Synthetic
TFTFTFTFTFTGGSDPGYIGSR

SEQ ID NO: 75 - protein, 23 aa, Artificial Sequence
  Synthetic
TFTFTFTFTFTGGRNIAELLKDI

SEQ ID NO: 76 - protein, 23 aa, Artificial Sequence
  Synthetic
TFTFTFTFTFTPRGDSGYRGDSG

SEQ ID NO: 77 - protein, 11 aa, Unknown
  Mammalian
PRGDSGYRGDS

SEQ ID NO: 78 - protein, 15 aa, Unknown
  Mammalian
KLTWQELYQLKYKGI

(19) United States
(12) Patent Application Publication
(10) Pub. No.: US 2011/0200560 A1
(43) Pub. Date: Aug. 18, 2011

(54) NON-IONIC SELF-ASSEMBLING PEPTIDES AND USES THEREOF

(75) Inventor: Shuguang Zhang, Lexington, MA (US)

(73) Assignee: MASSACHUSETTS INSTITUTE OF TECHNOLOGY, Cambridge, MA (US)

(21) Appl. No.: 12/787,546

(22) Filed: May 26, 2010

Related U.S. Application Data
(60) Provisional application No. 61/181,034, filed on May 26, 2009.

(51) Int. Cl. A61K 35/00; C07K 7/08; C07K 7/06; C07K 14/00; A61P 17/02; C12N 5/071; B82Y 30/00; B82Y 40/00

(52) U.S. Cl. 424/93.1; 530/326; 530/327; 530/328; 530/324; 530/325; 435/395; 977/700; 977/882

(57) ABSTRACT

The invention relates to a novel class of non-ionic, self-assembling peptides, compositions thereof, methods for the preparation thereof and methods of use thereof.

### RELATED APPLICATION

This application claims the benefit of U.S. Provisional Application No. 61/181,034 filed on May 26, 2009. The entire teachings of the above application are incorporated herein by reference.

### GOVERNMENT SUPPORT

The invention was supported, in whole or in part, by Grant No. EB003805 from the National Institutes of Health. The Government has certain rights in the invention.

### BACKGROUND OF THE INVENTION

Molecular self-assembly is the spontaneous organization of molecules into structurally well-defined arrangements due to non-covalent interactions. The resulting supramolecular structure usually provides nanoarchitectures with defined macroscopic properties. Several peptide molecular self-assembly systems have been developed. Most previously described self-assembling peptides are β-sheet peptides with alternating positively and negatively charges and various hydrophobic residues. The ionic complementary side chains of self-assembling peptides have been classified into several moduli (e.g., modulus I-IV) based on the hydrophilic surface of the molecules. Self-assembling peptides have been described as having utility in numerous applications, including scaffolding for tissue repair and regenerative medicine, drug delivery, three dimensional (3D) tissue culture as well as biological surface engineering.

It would be advantageous to design additional classes of self-assembling peptides.

### SUMMARY OF THE INVENTION

The invention relates to a novel class of non-ionic, self-assembling peptides, compositions thereof, methods for the preparation thereof and methods of use thereof.

In one embodiment, the invention is directed to an isolated peptide comprising a self-assembling motif, wherein the self-assembling motif has an amino acid sequence having a formula selected from the group consisting of:

[(τ)ₖ(Φ)ₚ]ₘ(τ)ₙ (Formula I); and

[(Φ)ₚ(τ)ₖ]ₘ(Φ)ₙ (Formula II);

wherein each (τ) is independently a non-ionic, polar amino acid;
wherein each (Φ) is independently a hydrophobic amino acid;
wherein k is an integer greater than or equal to 1;
wherein p is an integer greater than or equal to 1;
wherein m is an integer greater than or equal to 3;
wherein n is 0 or 1;
wherein each amino acid is a D-amino acid or each amino acid is a L-amino acid.

In one embodiment, (Φ) is a natural amino acid and (τ) is a natural amino acid. In another embodiment, (Φ) is a non-natural amino acid and (τ) is a non-natural amino acid.

In another embodiment, k is an integer between 1 and 10. In a further embodiment, p is an integer between 1 and 10. In another embodiment, k is an integer between 1 and 5. In a further embodiment, p is an integer between 1 and 5. In an additional embodiment, k is an integer between 1 and 10 and p is an integer between 1 and 10. In another embodiment, k is 1. In yet another embodiment, p is 1. In an additional embodiment, k is 1 and p is 1.

In yet another embodiment, the self-assembling peptide comprises a self-assembling motif having a formula selected from Formula (I) or (II), wherein each (Φ) is a hydrophobic amino acid independently selected from the group consisting of alanine, valine, isoleucine, leucine and phenylalanine.

In an additional embodiment, the self-assembling peptide comprises a self-assembling motif having a formula selected from Formula (I) or (II), wherein each (τ) is independently selected from the group consisting of serine, threonine, asparagine, and glutamine, tyrosine and hydroxy-proline.

In another embodiment, each (τ) is independently selected from the group consisting of serine or threonine.

In a further embodiment, the self-assembling peptide comprises a self-assembling motif having a formula selected from Formula (I) or (II) wherein (τ) is the same non-ionic, polar amino acid. In an additional embodiment, (τ) is serine, threonine, tyrosine and hydroxy-proline. In a further embodiment, the self-assembling peptide comprises a self-assembling motif having a formula selected from Formula (I) or (II) wherein each (Φ) is the same hydrophobic amino acid.

In a further embodiment, the self-assembling peptide comprises a self-assembling motif having a formula selected from Formula (I) or (II) wherein the peptide comprises a biologically active motif.

In an additional embodiment, the invention is directed to an aqueous composition comprising a plurality of peptides comprising a self-assembling motif, wherein each self-assembling motif has the amino acid sequence of Formula (I) or (II) and wherein said peptides are capable of self-assembly. In one embodiment, the aqueous composition is a homogenous mixture of peptides. In another embodiment, the aqueous composition comprises a heterogenous mixture of peptides.

In another embodiment, the invention is a self-assembled nanostructure, wherein the nanostructure comprises a peptide comprising a self-assembling motif. In some aspects, the nanostructure comprises a peptide comprising a self-assembling motif having an amino sequence of Formula (I) or (II).

In yet another embodiment, the invention is a method of preparing a self-assembled nanostructure comprising forming an aqueous mixture of peptides comprising a self-assembling motif under conditions suitable for self-assembly of the peptides. In some aspects, the nanostructure comprises a peptide comprising a self-assembling motif having an amino sequence of Formula (I) or (II).

In a further embodiment, the invention is a macroscopic material comprising a plurality of peptides, wherein each peptide comprises a self-assembling motif having an amino sequence of Formula (I) or (II) and wherein said peptides are capable of self-assembly. In some aspects, the material is composed of n-sheets. In additional aspects, the invention is a method for in vitro cell culture comprising adding a macroscopic membrane of the invention to a cell culture medium comprising cells, thereby forming a membrane/culture mixture; and b) maintaining the mixture under conditions sufficient for cell growth.

In yet another embodiment, the invention is a macroscopic scaffold comprising a plurality self-assembling peptides, wherein said peptides comprise a self-assembling motif having an amino acid sequence of Formula (I) or (II), wherein said peptides self-assemble into a β-sheet macroscopic scaffold; and wherein said macroscopic scaffold encapsulates living cells, said cells being present in said macroscopic scaffold in a three-dimensional arrangement.

In an additional aspect, the invention is a method of regenerating a tissue, said method comprising administering to a mammal a solution comprising self-assembling peptides and living cells; wherein said peptides comprise a self-assembling motif having an amino acid sequence selected of Formula (I) or (II), wherein said peptides do not substantially self-assemble prior to said administration, and wherein said peptides self-assemble into a β-sheet macroscopic scaffold after said administration, thereby encapsulating said cells in vivo, said cells being present in said macroscopic scaffold in a three-dimensional arrangement.

These and other aspects of the invention, as well as various advantages and utilities, will be more apparent with reference to the drawings and the detailed description of the embodiments of the invention.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A shows the self-assembling peptide nanofiber scaffold hydrogel. Numerous individual peptides in water self-assemble into a single nanofiber that further self-organize into a scaffold with greater than 99% water content.

FIG. 1B shows molecular models of the ionic peptides RADA 16-I, RADA 16-II, EAK 16-I and EAK 16-II.

FIG. 2 shows scanning electron microscope (SEM) images of nanofiber scaffolds made from ionic self-assembled peptides. (A) Matrigel where the nanofibers are not smooth with small particles. (B) The self-assembling, ionic peptide RADA16 where the nanofibers are smooth since there is no impurity and every ingredient is known in the scaffold. The scaffold is self-assembled from numerous identical 16-residue RADA16 peptides.

FIG. 3 shows SEM images of adult mouse neural stem cells fully embedded in self-assembling peptide nanofiber scaffold made from an ionic self-assembled peptide. The cells also produced their own extracellular matrix in nanoscale. The scale of peptide scaffold and extracellular matrix are indistinguishable. The cells can proliferate and differentiate very well in the microenvironment.

FIG. 4 shows human umbilical vein endothelial cell (HUVEC) unidirectional migration in response to functional peptide scaffolds made from an ionic self-assembled peptide using the clear boundary sandwich cell migration assay. Contrast microscopy images of HUVECs seeded on ionic, peptide scaffolds: A) RAD/PRG; B) RAD/KLT; C) and D) RADA 16-I, and fluorescent green nuclear staining for E) RAD/PRG; F) RAD/KLT; G) and H) RADA 16-I. Cells directionally migrated from RADA 16-I to RAD/PRG (C and G) and RAD/KLT (D and H). The scale bar is 100 um for all panels.

FIG. 5 shows molecular and schematic models of the designer self-assembling peptide nanofiber scaffold made from ionic self-assembled peptides. A) Molecular model of a single nanofiber with various incorporated active motifs. The hydrophobic residues, alanine, valine, isoleucine and phenylalanine facilitate self-assembly in water. Various active motifs are directly extended from self-assembling motifs with 1-2 glycine spacers. Furthermore, various proteins can also be linked onto the designer scaffold. B) Schematic model of the several different functional motifs (different colored bars and balls) could be extended from self-assembling motifs (backbone). These peptides can be combined in different ratios. A schematic model of a self-assembling nanofiber scaffold with combinatorial motifs carrying different biological functions is depicted.

### DETAILED DESCRIPTION OF THE INVENTION

A description of preferred embodiments of the invention follows.

The words “a” or “an” are meant to encompass one or more, unless otherwise specified. For example, the term “a peptide” encompass one or more peptides unless indicated otherwise.

The term “self-assembly” is a process of molecules or peptides forming regular shaped structures or aggregates in response to conditions in the environment, such as when added to an aqueous medium.

The term “self-assembling peptide” refers to a peptide comprising a self-assembling motif. Self-assembling peptides are peptides that are capable of self-assembly into structures including, but not limited to, macroscopic membranes, nanostructures and the like. Various self-assembling peptides and methods for preparation thereof have been described previously, for example in, U.S. Pat. Nos. 5,670,483, 5,955,343, 6,368,877, 7,098,028, 7,179,784 and 7,449,180 and U.S. Patent Application Publication No's. 2005/0181973 and 2007/0203062, the contents of each of which are herein incorporated by reference.

The term “self-assembling motif” refers to a peptide sequence or motif capable of self-assembly.

The term “ionic self-assembling peptide” refers to a self-assembling peptide comprising an alternating sequence of hydrophobic amino acids and hydrophilic amino acids, wherein said hydrophilic amino acids are charged amino acids. In some embodiments, the ionic self-assembling peptide comprises an alternating sequence of hydrophobic amino acids and hydrophilic amino acids, wherein said hydrophilic amino acids are acidic or basic amino acids. Ionic, self-assembling peptides have been described for example in U.S. Pat. No. 5,670,483.

As used herein, the term “amino acid” encompasses a naturally or non-naturally occurring amino acid. Non-naturally occurring amino acids are also referred to herein as “non-natural amino acids.” Naturally occurring amino acids are also referred to herein as “natural amino acids.” Natural amino acids are represented by their well-known single-letter designations: A for alanine, C for cysteine, D for aspartic acid, E for glutamic acid, F for phenylalanine, G for glycine, H for histidine, I for isoleucine, K for lysine, L for leucine, M for methionine, N for asparagines, P for proline, Q for glutamine, R for arginine, S for serine, T for threonine, V for valine, W for tryptophan and Y for tyrosine.

The term “physiologic pH” is a pH of about 7.

The term “scaffold” refers to a three-dimensional structure capable of encapsulating a guest molecule, including but not limited to, a cell or a pharmacologic agent.

The term “macroscopic” means having dimensions large enough so as to be visible under magnification of 10-fold or less. A macroscopic structure can be two-dimensional or three-dimensional. The terms “macroscopic structure” and “macroscopic material” are used interchangeably herein.

A “biologically active peptide motif” or “biologically active motif” or “biologically active domain” is a peptide motif that induces a phenotypic response or change in an appropriate cell type when the cell is contacted with the peptide comprising the biologically active motif. Biologically active motifs have been described, for example, U.S. Pat. No. 7,713,923, the contents of which are expressly incorporated by reference herein. In some aspects, a biologically active motif is a motif found in a naturally occurring protein. The biologically active peptide motif can be present in isolated form or as part of a larger polypeptide or other molecule. The ability of the peptide to elicit the response can be determined, for example, by comparing the response in the absence of the peptide (e.g., by mutating or removing the peptide when normally present within a larger polypeptide). In some embodiments of the invention, phenotypic responses or changes include, but are not limited to, enhancement of cell spreading, attachment, adhesion, proliferation, secretion of an extracellular matrix (ECM) molecule, or expression of a phenotype characteristic of a particular differentiated cell type.

As used herein, “isolated” means 1) separated from at least some of the components with which it is usually associated in nature or which naturally accompany it; 2) prepared or purified by a process that involves the hand of man; and/or 3) not occurring in nature.

According to the present invention, a “peptide”, “polypeptide”, or “protein” comprises a sequence of at least two amino acids linked together by peptide bonds. Amino acid sequences and formulae described herein are written according to convention such that the sequences are read from left to right wherein the left corresponds to the N-terminal end and the right corresponds to the C-terminal end.

In one embodiment, the amino acids of the inventive self-assembling peptides are natural amino acids. In another embodiment, the amino acids of the self-assembling motif comprise a non-natural amino acid. Numerous classes of non-natural amino acids including D-amino acids have been described (Luo et al. (2008), PLoS ONE 3(5): e2364. doi:10.1371/journal.pone.0002364, the contents of which are incorporated by reference herein). An exemplary, non-natural amino acid is hydroxy-proline. In another embodiment, all amino acids within the self-assembling peptide are L-amino acids. In an additional embodiment, all amino acids within the self-assembling peptide are D-amino acids. In other embodiments, one or more of the amino acids in an inventive peptide can be altered or derivatized, for example, by the addition of a chemical entity such as an acyl group, a carbohydrate group, a carbohydrate chain, a phosphate group, a farnesyl group, an isofarnesyl group, a fatty acid group, a linker for conjugation or functionalization, etc.

The peptides self-assemble when they form hydrogen bonds between adjacent polar side chains and when the peptides are structurally compatible. Structurally compatible peptides are peptides capable of maintaining a sufficiently constant intra-peptide distance to allow structure formation. It is however also contemplated that larger variations in the intra-peptide distance may not prevent structure formation if sufficient stabilizing forces are present. The intra-peptide distance can be calculated for each hydrogen bonding pair by taking the sum of the number of unbranched atoms on the side-chains of each amino acid in the pair. A mixture or plurality of peptides is referred to as “capable of self-assembly” when they are capable of forming hydrogen bonds between adjacent polar side chains and when they are structurally compatible.

The self-assembling motif described herein comprises alternating sequences of hydrophobic and non-ionic, polar amino acids. An example of an alternating sequence of hydrophobic and non-ionic, polar amino acid is a sequence in which one or more hydrophobic amino acids is followed by one or more non-ionic polar amino acids which in turn is followed by one or more non-ionic polar amino acids, etc., to the C-terminal end of the motif. In certain aspects of the invention, the self-assembling motif comprises at least 6 amino acids. It is anticipated that the longer the self-assembling motif, the more likely the peptide is to self-assemble.

It has been found that the more hydrophobic the amino acids within the self-assembling motif are, the more readily the peptide undergoes self-assembly. Exemplary hydrophobic amino acids that can be used according to the invention are glycine, alanine, leucine, isoleucine, phenylalanine tyrosine, and tryptophan. The hydrophobic amino acids of the self-assembling motif can all be the same or can be different. Non-limiting examples of self-assembling motifs in which the hydrophobic amino acids are different is where one hydrophobic amino acid is different from the other hydrophobic amino acids or where two hydrophobic amino acids are different from the other hydrophobic amino acids or wherein some amino acids are different from the other amino acids or where all of the hydrophobic amino acids are different from each other, etc. In one embodiment, each (Φ) is a hydrophobic amino acid independently selected from the group consisting of alanine, leucine, isoleucine, phenylalanine, tyrosine and tryptophan. In another embodiment, each (Φ) is a hydrophobic amino acid independently selected from the group consisting of alanine, valine, leucine, isoleucine and phenylalanine. In a further embodiment, each (Φ) is the same hydrophobic amino acid, wherein the hydrophobic amino acid is selected from the group consisting of alanine, leucine, isoleucine, phenylalanine, tyrosine and tryptophan.

Non-ionic, polar amino acids are those amino acids with non-ionic, polar sidechains. Exemplary non-ionic, polar amino acids are threonine, serine, asparagine, glutamine, tyrosine, and hydroxy-proline. The non-ionic, polar amino acids of the self-assembling motif can all be the same or can be different. Non-limiting examples of self-assembling motifs where the non-ionic, polar amino acids are different is where one non-ionic, polar amino acid is different from the other non-ionic, polar amino acids or where two non-ionic, polar amino acids are different from the other non-ionic, polar amino acids or where some non-ionic polar amino acids are different from other non-ionic polar amino acids or where all of the non-ionic polar amino acids are different from each other, etc. In one embodiment, each (τ) is a non-ionic, polar amino acid independently selected from the group consisting of thereonine, serine, asparagine and glutamine. In another embodiment, each (τ) is a non-ionic, polar amino acid independently selected from the group consisting of threonine and serine. In a further embodiment, each (τ) is the same non-ionic, polar amino acid wherein the non-ionic polar amino acid is selected from the group consisting of threonine, serine, asparagines and glutamine. In an additional embodiment, each (τ) is the same non-ionic, polar amino acid wherein the non-ionic, polar amino acid is selected from the group consisting of threonine and serine. In another embodiment, each (τ) is the same non-ionic polar amino acid, wherein the non-ionic polar amino acid is threonine.

Exemplary self-assembling peptides are shown below in Table 1.

TABLE 1

| SEQ ID | | Number of |
|---|---|---|
| NO | Sequence | Residues |
| 1 | ATATATATATATATATA | 17 |
| 2 | VTVTVTVTVTVTVT | 14 |
| 3 | VTVTVTVTVTVT | 12 |
| 4 | TITITITITITIT | 13 |
| 5 | TITITITITIT | 11 |
| 6 | TFTFTFTFTFT | 11 |
| 7 | TFTFTFTFT | 9 |

In another embodiment of the invention, the C-terminus of the peptide is amidated. In yet another embodiment, the N-terminus of the self-assembling peptide is acetylated. Exemplary self-assembling peptides of the invention wherein the C-terminus is amidated and the N-terminus is acetylated are shown below in Table 2.

TABLE 2

| SEQ | | | Number |
|---|---|---|---|
| ID | | | of |
| NO | Name | Sequence | Residues |
| 8 | TATA17 | Ac-ATATATATATATATATA-NH2 | 17 |
| 9 | TVTV14 | Ac-VTVTVTVTVTVTVT-NH2 | 14 |
| 10 | TVTV12 | Ac-VTVTVTVTVTVT-NH2 | 12 |
| 11 | TITI13 | Ac-TITITITITITIT-NH2 | 13 |
| 12 | TITI11 | Ac-TITITITITIT-NH2 | 11 |
| 13 | FTFT11 | Ac-TFTFTFTFTFT-NH2 | 11 |
| 14 | FTFT9 | Ac-TFTFTFTFT-NH2 | 9 |

In yet another embodiment, the self-assembling motif has an amino sequence having a formula selected from the group consisting of:

[(τ₁)(Φ)(τ₂)(Φ)]_{q}(τ₁)ₙ (Formula III);

[(Φ)(τ₁)(Φ)(τ₂)]_{q}(Φ)ₙ (Formula IV);

[(τ₁)(Φ)(τ₂)(Φ)]_{q}(τ₁)ₙ (Formula V); and

[(Φ)(τ₂)(Φ)(τ₁)]_{q}(Φ)ₙ (Formula VI);

wherein (τ₁) is serine;

wherein (τ₂) is threonine;

wherein each (Φ) is independently a hydrophobic amino acid;

wherein q is an integer greater than or equal to 2;

wherein n is 0 or 1;

wherein each amino acid is a D-amino acid or each amino acid is a L-amino acid.

In one embodiment, the self-assembling motif has a formula selected Formulae (III) to (VI) and each (Φ) is the same hydrophobic amino acid.

The self-assembling peptides described herein comprise a self-assembling motif. In another embodiment, the self-assembling peptide consists essentially of a self-assembling motif described herein. In yet another embodiment, the self-assembling peptide consists of a self-assembling motif described herein. In a further embodiment, the self-assembling peptide has the amino acid sequence of Formula (I) to (VI).

In addition to one or more self-assembling motifs, the peptides can additionally comprise a second amino acid domain or motif that would not self-assemble if present in isolated form (referred to herein as the “second amino acid domain” or “second amino acid motif”). Self-assembling peptides comprising an amino acid domain that does not self-assemble when present in isolated form has been described, for example, in U.S. Pat. App. Pub. No. 2005/0181973, the contents of which are incorporated by reference herein. In certain embodiments of the invention, the conditions under which self-assembly of the self-assembling peptide comprising a self-assembling motif and a second amino acid domain occurs are the same as the conditions under which the corresponding peptide in the absence of the second amino acid domain. The self-assembling motif and the second amino acid domain can be joined, for example, via a linker or bridge. In one embodiment the linker or bridge is one or more amino acids or a different molecular entity. In another embodiment, the linker domain consists of one or more glycine (G) residues, e.g., 1, 2, 3, 4, 5, etc. glycines, may be used. Glycine is small, nonpolar amino acid and is thus unlikely to substantially interfere with self-assembly. In other embodiments, an amino acid with a non-polar side chain is used as the linker.

In some embodiments of the invention, the second amino acid domain is a biologically active peptide motif. Biologically active motifs that can be used according to the present invention include those described in U.S. Pat. App. Pub. No. 2005/0181973 and in Gelain et al. (2006). PLoS ONE 1(1): e119. doi:10.1371/journal.pone.0000119, the contents of which are incorporated by reference herein. Biologically active motifs include, for example, short peptide sequences from proteins in the cellular basement membrane that have been identified as participating in several biological functions such as cell attachment, proliferation, differentiation and migration (Iwamoto et al. (1987). Science 238: 1132-34; Kleinman et al. (1989), PNAS 87: 2279-83; Koliakos et al. ((1989), J. Biol. Chem. 264, 2313-2323). Various biologically active motifs have been identified, for example, in laminin, collagen IV, nidogen, proteoglycans and vascular endothelial cells. For example, the sequence AASIKVAVSADR (SEQ ID NO: 15) derived from the laminin α chain promoted activity of neurite extension by PC12 cells, the peptide CSRARKQAASIKVAVSADR (SEQ ID NO: 16) induced degradation of Matrigel matrix by human umbilical vein endothelial cells (HIVEC). In addition, the sequences YIGSR (SEQ ID NO: 17), PDSGR (SEQ ID NO: 18) and RYVVLPR (SEQ ID NO: 19) located on the β1 chain of laminin promoted cell adhesion. The sequence KAFDITYVRLKF (SEQ ID NO: 20) from the laminin γ1 chain also promoted HUVEC adhesion and tube formation, as well as neuronal cell adhesion and neurite outgrowth. The peptide sequence TAGSCLRKFSTM (SEQ ID NO: 21) from type IV collagen was found to specifically bind to heparin. Additionally, RGD sequences, found, for example, in nidogen, serve as cell attachment site.

Non-limiting examples of biologically active motifs that can be used according to the invention are shown in Table 3 below.

TABLE 3

| SEQ | | |
|---|---|---|
| ID | | |
| NO | Peptide sequence | Protein |
| 15 | AASIKVAVSADR | Laminin-1 |
| 16 | CSRARKQAASIKVAVSADR | Laminin-1 |
| 17 | YIGSR | Laminin-1 |
| 18 | PDGSR | Laminin-1 |
| 19 | RYVVLPR | Laminin-1 |
| 20 | KAFDITYVRLKF | Laminin-1 |
| 21 | TAGSCLRKFSTM | Collagen IV |
| 22 | RNIAEIIKDI | Laminin-1 |
| 23 | YVRL | Laminin-1 |
| 24 | IRVTLN | Laminin-1 |
| 25 | TTVKYIFR | Laminin-1 |
| 26 | SIKIRGTY | Laminin-1 |
| 27 | RQVFQVAYIIIKA | Laminin-1 |
| 28 | FQIAYVIVKA | Laminin-1 |
| 29 | GQLFHVAYIIIKA | Laminin-1 |
| 30 | FHVAYVLIKA | Laminin-1 |
| 31 | LENGEIVSLVNGR | Laminin-1 |
| 32 | LGTIPG | fibronectin |
| 33 | DGEA | fibronectin |
| 34 | REDV | fibronectin |
| 35 | GVGVP | elastin |
| 36 | GVGVAP | elastin |
| 37 | IKVAV | Laminin |
| 38 | PFSSTKT | Bone marrow homing |
| | | peptide 2 |
| 39 | SKPPGTSS | Bone marrow homing |
| | | peptide 1 |
| 40 | SDPGYIGSR | laminin |
| 41 | RNIAELLKDI | Laminin-1 |
| 42 | PRGDSGYRGDSG | Collagen IV |
| 43 | GFLGFPT | myelopeptide |
| 44 | YGPDSGR | Laminin-1 |

Exemplary self-assembling peptides comprising biologically active motifs are shown below in Table 4.

TABLE 4

| SEQ ID | | | Number of |
|---|---|---|---|
| NO | Name | Sequence | Residues |
| 45 | aPDS | Ac-ATATATATATATATATAYGPDSGR-NH2 | 25 |
| 46 | aIKV | Ac-ATATATATATATATATAGIKVAV-NH2 | 23 |
| 47 | aYIG | Ac-ATATATATATATATATAGYIGSR-NH2 | 23 |
| 48 | aPFS | Ac-ATATATATATATATATAGPFSSTKT-NH2 | 25 |
| 49 | aSKP | Ac-ATATATATATATATATAGSKPPGTSS-NH2 | 26 |
| 50 | aSDP | Ac-ATATATATATATATATAGGSDPGYIGSR-NH2 | 28 |
| 51 | aRNI | Ac-ATATATATATATATATAGGRNIAELLKDI-NH2 | 29 |
| 52 | aPRG | Ac-ATATATATATATATATAGPRGDSDGYRGDSG-NH2 | 30 |
| 53 | vPDS | Ac-VTVTVTVTVTVTVTGYGPDSGR-NH2 | 22 |
| 54 | vIKV | Ac-VTVTVTVTVTVTVTGIKVAV-NH2 | 20 |
| 55 | vYIG | Ac-VTVTVTVTVTVTVTGYIGSR-NH2 | 20 |
| 56 | vPFS | Ac-VTVTVTVTVTVTVTGPFSSTKT-NH2 | 22 |
| 57 | vSKP | Ac-VTVTVTVTVTVTVTGSKPPGTSS-NH2 | 23 |
| 58 | vSDP | Ac-VTVTVTVTVTVTVTGGSDPGYIGSR-NH2 | 25 |
| 59 | vRNI | Ac-VTVTVTVTVTVTVTGGRNIAELLKDI-NH2 | 26 |
| 60 | vPRG | Ac-VTVTVTVTVTVTVTGPRGDSGYRGDSG-NH2 | 27 |
| 61 | iPDS | Ac-TITITITITITITGYGPDSGR-NH2 | 21 |
| 62 | iIKI | Ac-TITITITITITITGIKVAV-NH2 | 19 |
| 63 | iYIG | Ac-TITITITITITITGYIGSR-NH2 | 19 |
| 64 | iPFS | Ac-TITITITITITITGPFSSTKT-NH2 | 21 |
| 65 | iSKP | Ac-TITITITITITITGSKPPGTSS-NH2 | 22 |
| 66 | iSDP | Ac-TITITITITITITGGSDPGYIGSR-NH2 | 24 |
| 67 | iRNI | Ac-TITITITITITITGGRNIAELLKDI-NH2 | 25 |
| 68 | iPRG | Ac-TITITITITITITGPRGDSGYRGDSG-NH2 | 26 |
| 69 | fPDS | Ac-TFTFTFTFTFTGYGPDSGR-NH2 | 19 |
| 70 | fIKV | Ac-TFTFTFTFTFTGIKVAV-NH2 | 17 |
| 71 | fYIG | Ac-TFTFTFTFTFTGYIGSR-NH2 | 17 |
| 72 | fPFS | Ac-TFTFTFTFTFTGPFSSTKT-NH2 | 19 |
| 73 | fSKP | Ac-TFTFTFTFTFTGSKPPGTSS-NH2 | 20 |
| 74 | fSDP | Ac-TFTFTFTFTFTGGSDPGYIGSR-NH2 | 22 |
| 75 | fRNI | Ac-TFTFTFTFTFTGGRNIAELLKDI-NH2 | 23 |
| 76 | fPRG | Ac-TFTFTFTFTFTPRGDSGYRGDSG-NH2 | 24 |

In addition to amino acid domains whose sequences are derived from naturally occurring proteins such as those mentioned above, amino acid domains from growth factors, cytokines, chemokines, peptide hormones, peptide neurotransmitters, other biologically active peptides found in the body, etc., can also be used (See, for example, Goodman and Gilman, The Pharmacological Basis of Therapeutics, 10th Ed. McGraw Hill, 2001 and Kandel et al., Principles of Neural Science, 4th ed., McGraw Hill, 2000).

Other biologically active motifs can be identified according to a number of criteria including, but not limited to, those described by Yamada (1991), J. Biol. Chem, 266: 12809-12812. While Yamada describes tests for biological relevance for the case of adhesive recognition sequences, the criteria given may be applied more widely. A putative active peptide motif can be identified as biologically active when a synthetic peptide containing the sequence displays activity after conjugation to a carrier (e.g., IgG, albumin, beads), even if inactive when adsorbed directly on a substrate such as glass, plastic, etc., though they may also display activity when conjugated to a substrate. A soluble form of a biologically active peptide motif can competitively inhibit the function of an intact protein in which the motif is naturally found. Alteration of the peptide sequence can eliminate the function of the peptide. A biologically active peptide can bind to the same cellular receptor or naturally occurring biomolecule as a naturally occurring protein containing the peptide. A range of different peptide concentrations can be tested, and various combinations can be used.

The inventive self-assembling peptides self-assemble in aqueous solution. The rate of self-assembly can be increased by exposure to a sufficient concentration of a monovalent cation. Exemplary monovalent cations are Li+, Na+, K+ and Ca+. The self-assembled structures are stable in aqueous compositions, for example, in water, phosphate-buffered saline (PBS), tissue culture medium, serum, and also in ethanol, and can be transferred to and stored in any of these media. In one embodiment, the invention is directed to an aqueous composition comprising a plurality of self-assembling peptides described herein. As used herein, a plurality of self-assembling peptides is at least two self-assembling peptides. The plurality of self-assembling peptides can be a homogenous mixture of peptide or can be a heterogenous mixture of peptides. A homogenous mixture of self-assembling peptides is a mixture wherein each of the self-assembling peptides has the same peptide sequence and are capable hydrogen bonding between polar side chains and structurally compatible, thus capable of self-assembly. A heterogenous mixture of self-assembling peptides is a mixture comprising at least two self-assembling peptides which can form hydrogen bonds between their respective polar side chains and which are structurally compatible. In one embodiment, the invention is a nanostructure comprising a plurality of self-assembling peptides of the invention. Exemplary nanostructures include nanofilaments, nanofibers and nanoscaffolds.

In another embodiment, the invention is a macroscopic structure comprising a plurality of self-assembling peptides of the invention. In one embodiment, the macroscopic structure comprises homogenous self-assembling peptides. In another embodiment, the macroscopic structure comprises heterogenous self-assembling peptides. The term “homogenous self-assembling peptides” refers to a plurality of identical or same self-assembling peptides. The term “heterogenous self-assembling peptides” refers to a plurality of different self-assembling peptides. In some embodiments, the macroscopic structure is a macroscopic membrane. The macroscopic membranes comprise, for example, a plurality of interwoven nanofilaments which in turn comprise the inventive self-assembling peptides. The initial concentration of the peptide is a factor in the size and thickness of the membrane formed. In general, the higher the peptide concentration, the higher the extent of membrane formation. In a further embodiment, the macroscopic membrane is the form of β-sheets. In one embodiment, the invention is a macroscopic scaffold comprising a plurality of self-assembling peptides comprising a self-assembling motif of Formula (I) or (II), wherein the self-assembling peptides self-assemble into a β-sheet macroscopic scaffold. It has been shown that β-branched amino acids have a greater tendency to form β-sheet structures. Therefore, in some embodiments, the hydrophobic amino acid is a β-branched amino acid. Exemplary β-branched hydrophobic amino acids are valine and isoleucine. In other aspects, the non-ionic, polar amino acid is a β-branched amino acid. An exemplary β-branched non-ionic, polar amino acid is threonine.

The formation of a self-assembled structure can be observed with the naked eye after staining with a Congo Red, a dye which preferentially stains β-sheet structures and is commonly used to visualize abnormal protein deposition in tissues. Additional structural details can be observed under magnification. Scanning electron microscopy (SEM) allows structural details of the nanostructures to be observed. The β-sheet secondary structures of the membranes can additionally be confirmed by circular dichroism (CD) spectroscopy (Zhang et al (1993), PNAS 90: 3334-8; Zhang et al (1995). Biomaterials 16:1385; Zhang & Rich 1997, PNAS 94, 23-28, Yokoi et al., 2005. PNAS 102, 8414).

In certain aspects, the invention encompasses methods of using the inventive self-assembling peptides and self-assembled structures thereof. The inventive self-assembling peptides are, for example, useful as cell culture supports, as self-assembled monolayers imprinted onto solid supports, for the repair and replacement of various tissues, as a scaffold to encapsulate living cells, as part of a controlled-release drug delivery system and for promoting hemostasis. The use of self-assembling peptides has been described, for example in, U.S. Pat. App. Pub. No's. 2002/0072074; 2002/0160471; 2004/0087013; 2004/0242469; 2005/0287186 and 2007/0203062, the contents of each of which are incorporated by reference herein.

In addition, because the macroscopic structure, macroscopic membranes and nanostructures formed by self-assembly of the inventive peptides are stable in serum, resistant to proteolytic digestion and alkaline and acidic pH, and are non-cytotoxic, the materials, membranes and filaments are useful in biomaterial applications, such as medical products (e.g., sutures), artificial skin or internal linings, slow-diffusion drug delivery systems supports for in vitro cell growth or culture and supports for artificial tissue for in vivo use. The structures can additionally be used in numerous applications in which permeable and water insoluble material are appropriate, such as separation matrices (e.g., dialysis membranes, chromatographic columns). Due to their permeability, the macroscopic membranes described herein are useful as slow-diffusion drug delivery vehicles. Because the membranes are resistant to degradation by proteases and stomach acid (pH 1.5), drug delivery vehicles made of these membranes could be taken orally. The small pore size of the membranes also makes them useful as filters, for example, to remove virus and other microscopic contaminants. The pore size (interfilament distance) and diameter of the filaments in the membranes can be varied by varying the length and sequence of the peptides used to form the membranes.

The macroscopic membranes can also be modified to give them additional properties. For example, the membranes can be further strengthened by cross-linking the peptides after membrane formation by standard methods. Collagen can be combined with the peptides to produce membranes more suitable for use as artificial skin; the collagen may be stabilized from proteolytic digestion within the membrane. Furthermore, combining phospholipids with the peptides may produce vesicles.

The macroscopic structures can also be useful for culturing cells. The addition of growth factors, such as fibroblast growth factor, to the peptide macroscopic structure can further improve attachment, cell growth and neurite outgrowth. The porous macrostructure of the biopolymers can also be useful for encapsulating cells. The pore size of the membrane can be large enough to allow the diffusion of cell products and nutrients. The cells are, generally, much larger than the pores and are, thus, contained. In another embodiment, the invention is a macroscopic scaffold comprising a plurality of self-assembling peptides of the invention, wherein the self-assembling peptides self-assemble into a β-sheet macroscopic scaffold and wherein said macroscopic scaffold encapsulates living cells and wherein said cells are present in said macroscopic scaffold in a three-dimensional arrangement. The macroscopic scaffold can be prepared by incubating the self-assembling peptides and living cells in an aqueous solution under conditions suitable for self-assembly. The invention, also encompasses a method of regenerating a tissue, the method comprising administering to a mammal a macroscopic scaffold comprising the inventive self-assembling peptides are self-assembled in a β-sheet macroscopic scaffold, wherein said macroscopic scaffold encapsulates living cells, said cells being present in said macroscopic scaffold in a three-dimensional arrangement. In another aspect, the invention features a method of regenerating a tissue which comprises administering to a mammal a macroscopic scaffold having amphiphilic peptides and encapsulated living cells. The encapsulated cells are present in the macroscopic scaffold in a three-dimensional arrangement. In some embodiments, the method is used to treat or prevent a cartilage defect, connective tissue defect, nervous tissue defect, epidermal lining defect, endothelial lining defect, or arthritis. The macroscopic scaffold can be administered orally, percutaneously, intramuscularly, intravenously, subcutaneously, or by any other appropriate mode. In another embodiment, the invention is a method for in vitro cell culture comprising: (a) adding a macroscopic membrane which is formed by self-assembly of the inventive self-assembling peptides in an aqueous solution to a cell culture medium comprising cells, thereby forming a membrane/culture mixture; (b) maintaining the mixture under conditions sufficient for cell growth.

In a further embodiment, the invention is a macroscopic scaffold comprising a plurality of self-assembling peptides of the invention, wherein the self-assembling peptides self-assemble into a β-sheet macroscopic scaffold, wherein said macroscopic scaffold encapsulates a living cell, wherein said cell is present in said macroscopic scaffold in a three-dimensional arrangement, wherein at least some self-assembling peptides comprise biologically active motifs wherein the biologically active motifs are capable of interacting with the cells. In another embodiment, the biologically active motifs are capable of binding to receptors on the cells.

The macroscopic membranes can also be used as a model system for investigating the properties of biological proteins structures with such unusual properties as extreme insolubility and resistance to proteolytic digestion including, but not limited to aggregates of β-amyloid protein and aggregated scrapie protein. The invention also encompasses method for the regeneration of nerves with the structures comprising self assembling peptides. For example, nerve regeneration can be promoted and directed by transplanting the self-assembled nanostructures along the correct path to their targets.

The following Example further illustrates the present invention but should not be construed as in any way limiting its scope.

### Example 1

### Designer Self-Assembling Peptides and Uses Thereof

Almost all adhering tissue cells have been studied using various soluble growth factors on coated 2-D flat platforms. The current paradigm emphasizes the profound influence of soluble factors on the ultimate cell fate [1-2]. However, there are 29 different collagens (at the latest count) and a wide range of extracellular matrices that also undoubtedly profoundly influence cell fate. For example, the extracellular matrix microenvironment of chondrocytes in cartilage and osteoblasts in bone is substantially different from that of the neural cells in brain. However, up to date, very few studies emphasize the biological engineered biologically active scaffold with minimal soluble growth factors.

Biomedical researchers have become increasingly aware of the limitations of conventional 2-D tissue cell cultures where most cell studies have been carried out. We are now searching and embracing 3-D cell culture systems, something between a Petri dish and a mouse. It becomes apparent that 3-D cell culture offers a more realistic microenvironment where the functional properties of cells can be observed and manipulated that is not possible in animals. The important implications of 3-D tissue cell cultures for basic cell biology, tumor biology, neurobiology and high-content drug screening are far-reaching. Quantitative biology requires in vitro culture systems that more authentically represent a cell's environment in a living organism. In doing so, in vitro experimentation can truly become more predictive of in vivo systems.

Nearly all tissue cells are embedded in 3-dimension (3-D) microenvironment in the body. On the other hand, nearly all tissue cells including most cancer and tumor cells, neural cells, and various types of stem cells have been studied in 2-dimension (2-D) platforms coated with various substrata. The architecture of the environment of a cell in a living organism is 3-D, cells are surrounded by other cells, where many extracellular ligands including many types of collagens, laminin, and other extracellular matrix proteins, not only allow attachments between cells and the basal membrane [3-5]; but also allow access to oxygen, hormones, and nutrients; removal of waste products and other cell types associated in tissues. The normal 3-D environment of cells consists of a complex network of extracellular matrix nanoscale fibers that create various local microenvironments.

There are several key drawbacks to 2D cell cultures. First, the movements of cells in the 3D environment of a whole organism typically follow a chemical signal or molecular gradient in X, Y and Z dimensions. Molecular gradients play a vital role in biological differentiation, determination of cell fate, organ development, signal transduction, neural information transmission and countless other biological processes [1-2]. Second, cells isolated directly from higher organisms frequently alter metabolism and alter their gene expression patterns when in 2D culture. It is clear that cellular structure plays a major role in determining cellular activity, through spatial and temporal extracellular matrix protein and cell receptor interactions that naturally exists in tissues and organs. The cellular membrane structure, the extracellular matrix and basement membrane significantly influences cellular metabolism, via the protein-protein interactions. The adaptation of cells to a 2D platform requires significant adjustment of the surviving cell population not only to changes in oxygen, nutrients and extracellular matrix interactions, but also to alter waste disposal. Third, cells growing in a 2-D environment significantly alter production of their own extracellular matrix and often undergo morphological changes (e.g., an increase in spreading). We asked the question of how realistic cell behavior is that does not take account of cellular communication, the transport of oxygen, nutrients, toxins, and cellular metabolism in the context of all 3 dimensions.

In the last few decades, biopolymers have been developed to culture cells in 3-D [6-7]. However, processed synthetic polymers consisting of microfibers ˜10-50 micrometers in diameter are similar in size to most cells. Thus, cells attached on microfibers are still in a 2-D environment with a curvature depending on the diameter of the microfibers. For a true 3-D environment, a scaffold's fibers and pores must be much smaller than the cells. In order to culture tissue cells in a truly 3-D microenvironment, the fibers must be significantly smaller than cells so that the cells are surrounded by the scaffold, similar to the extracellular environment and native extracellular matrices [8-9].

Animal-derived biomaterials (e.g., collagen gels, poly-glycosaminoglycans and MATRIGEL™) have been used as an alternative to synthetic scaffolds [10-21]. While they do have the right scale, they frequently contain residual growth factors, undefined constituents or non-quantified impurities. It is thus very difficult to conduct a completely controlled study using such materials because they vary from lot to lot. Although researchers are well aware of its limitation, it is one of the only few choices. Recently, more and more researchers have started to use 3D cell culture systems using a variety of new nanomaterials [22-26].

The described studies aim to systematically carry out biological engineering of biologically active designer self-assembling peptide nanofiber scaffolds to culture human neural progenitor cells using minimal soluble growth and differentiation factors. An ideal 3-D culture system should be fabricated from a synthetic biological material with defined constituents.

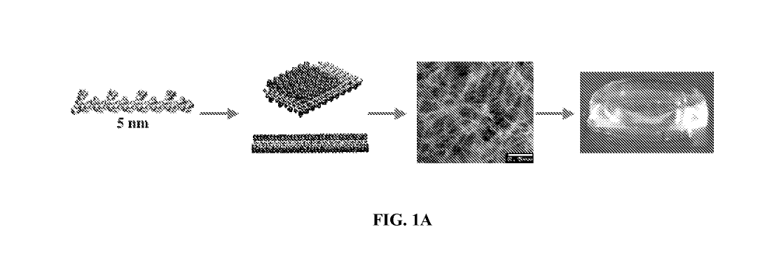

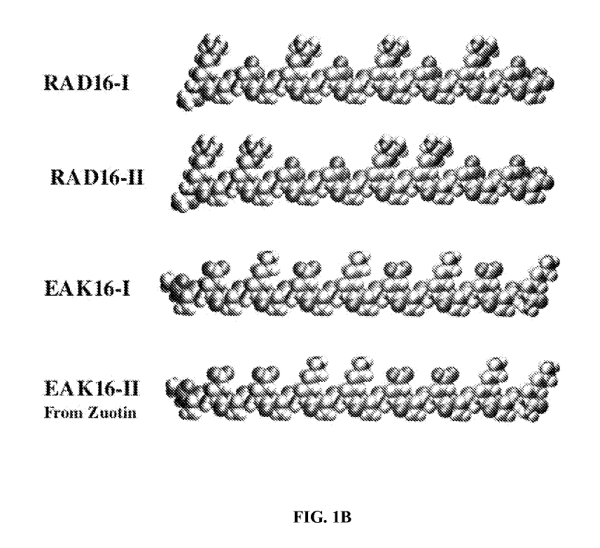

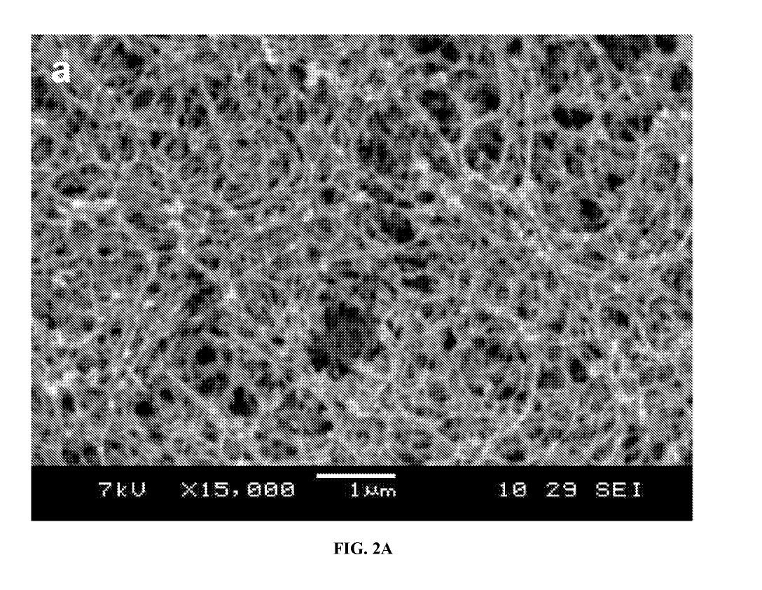

One of the first molecules of a class of designer self-assembling peptides to be discovered is EAK16-II, a 16 amino acid peptide that was found as a segment in a yeast protein, zuotin (zuo means left in Chinese), which was originally characterized by its ability to bind to left-handed Z-DNA [27]. Zuotin is a 433-residue protein of known sequence with a domain consisting of 34 amino acid residues (305-339) with alternating alanines and alternating charges of glutamates and lysines with an interesting regularity.[27]. We subsequently reported a class of biological materials made from these self-assembling peptides [28-30]. This biological scaffold consists of greater than 99% water content (peptide content 1-10 mg/ml) (FIG. 1). They form scaffolds when the peptide solution is exposed to physiological solutions including tissue culture media and salt solutions [31-33]. This is a unique property over other traditional biopolymer hydrogels, which require the catalysis of toxic chemicals and other initiators.

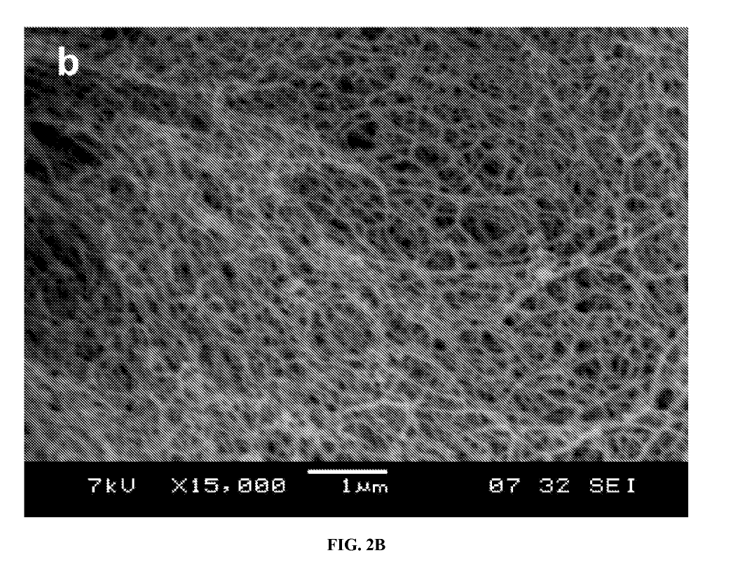

We demonstrated that peptides, made from natural amino acids, undergo self-assembly into well-ordered nanofibers and scaffolds, often ˜10 nm in diameter with pores between 5-200 nm [28-40]. These peptides can be chemically synthesized, designed to incorporate specific ligands, including ECM and other soluble growth factor ligands [1-10] for cell receptors, purified to homogeneity, and manufactured readily in large quantities. Their assembly into nanofibers can be controlled at physiological pH simply by altering salt concentrations. Because the resulting nanofibers are 1,000-fold smaller than synthetic polymer microfibers, they surround cells in a manner similar to extracellular matrix (FIG. 2). Moreover, biomolecules in such a nanoscale environment diffuse slowly and are likely to establish a local molecular gradient.

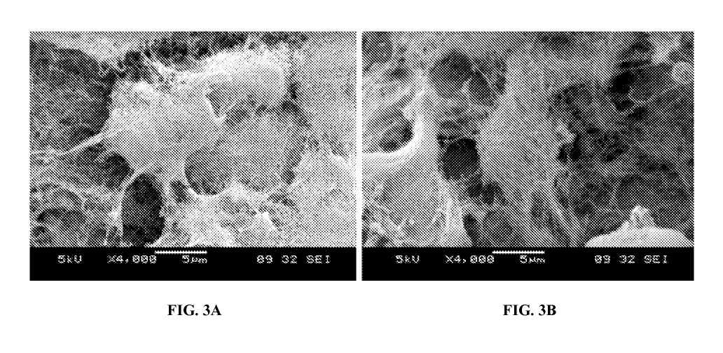

Using the designer synthetic self-assembling peptide nanofiber system, every ingredient of the scaffold can be defined, just as in a 2-D Petri dish; the only difference is that cells now reside in a 3-D environment (FIG. 3) where the extracellular matrix receptors on the cell surface can bind to the ligands on the peptide scaffold. Cells can now behave and migrate in a truly 3-D manner. Beyond the Petri dish, higher tissue architectures with multiple cell types, rather than monolayers, can also be constructed for tissues using the 3-D self-assembling peptide scaffolds.

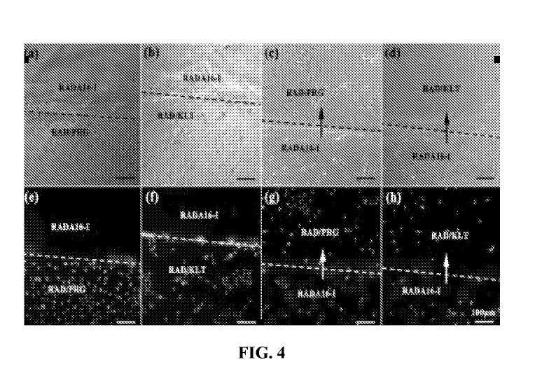

We also systematically compared a wide range of biomaterials and showed that peptide scaffolds are as good as or better than most other biomaterials [42]. Our results show high level of mouse neural stem cells differentiation toward both neuronal and glial phenotypes in the designer scaffolds in vitro serum-free condition without adding extra soluble growth factors [43]. These results are similar to those with Matrigel, a natural extract considered as the most effective and standard cell-free substrate for neural stem cells culture and differentiation. In the designer peptide scaffolds with functional motifs, not only mouse neural stem cell survival has been significantly improved, but it also enhanced their differentiation, when compared to the generic self-assembling peptides. Similar event was also observed in mouse pre-osteoblast MC3T3-E1cells in designer peptide scaffold 3-D cultures [44]. Furthermore, using the designer scaffold system, human umbilical vein endothelial cells showed very interesting unidirectional migration toward the scaffold with the active motifs without adding extra soluble growth factors [45]. In this particular experiment (FIG. 4), normal RADA16 peptide scaffold was one side and the same peptide containing active motif with 2 units RGD motif (PRGDSGYRGDS) (SEQ ID NO: 77) called PRG from its first 3 amino acid residues and another motif from vascular endothelial growth factor (KLTWQELYQLKYKGI) (SEQ ID NO: 78) called KLT (first 3 residues). These active scaffold induced cell migration without additional soluble growth factors [45]. In 3-D cell culture tissue models, one of important aspects is to establish a molecular gradient since cells often migrate toward a particular gradient during development and differentiation.

In addition, we have carried out slow molecular release experiment using the self-assembling peptide scaffold [46-47]. We showed that the peptide scaffolds not only slowly released small molecules, but also released protein molecules from lysozyme (˜14 Kd) to antibody (˜150 kd). The release profile for proteins is as function of the protein molecular size [47]. Thus it is highly likely that a molecular gradient will be established together in the 3D cell culture human tissue model system.

The peptide scaffold has been not only use for 3D cell cultures, but also used for many diverse experiments for tissue repairs [48-53]. These experiments include: 1) use the peptide to repair the optical nerve of hamster [48] and mouse spinal cord [49]. It also stopped bleeding in ˜10-15 seconds in various organs in animals [50]. The scaffold was also used to inject into mouse heart to improve cell therapy for myocardial infarction [51-53].

Built on our past research [55-59] and combining construction motifs of peptide and protein structural knowledge [1-10, 60-61], we will continue to design new scaffolds with biologically active motifs for 3D human neural tissue model.

Materials: a) Designer self-assembling non-ionic peptides will be synthesized b) NIH registered human cell lines. These cells are commercially available.
1) NHNP-Normal Human Neural Progenitors (Lonza, product number PT-2599).
2) The NIH registered human ES cell line (Invitrogen). All human ES cells are modified cells lines from NIH registered BG01V, such as BG01V/hoG which express GFP under oct4 promoter. These cell lines are not registered themselves.

b) Human neural stem cell culture media. All human stem cell culture media, growth and differentiation factors, specific antibodies and trace markers for assays, identifications, are also commercially available from Invitrogen, Lonza (BioWittaker), Millipore, Thermo Scientific (HyClone Cell Culture) R&D system, Sigma and elsewhere. For example: http://www.millipore.com/searchsummary.do?tabValue=&q=Stem+cell+culture+media http://www.invitrogen.com/site/us/en/home/Products-and-Services/Applications/Cel-Culture/Stem-Cell-Research/Neural-Stem-Cells.html.

Mouse neural stem cell 3D culture protocols using the self-assembling peptide scaffolds have been developed in my own laboratory (42-43), pre-osteoblast MC3T3-E1cells (44), human umbilical vein endothelial cells (45) and mouse neural cells from striatal tissue (62). More cells are also been used by others since the peptide scaffolds are now commercially available from BD Biosciences http://www.bdbiosciences.com/discovery_labware/products/display_product.php?keyID=179.

Many detail protocols for culture stem cells are widely available in literature and on the various websites, for example: http://ink.primate.wisc.edu/˜thomson/protocol.html#media, Also see a recent list in Nature, 451, 859, 14 Feb. 2008.

Since the discovery of the class of self-assembling peptides in 1992, the β-sheet peptides have been mostly designed with alternating positively (lysine & arginine) and negatively charges (aspartic acid & glutamic acid) and various hydrophobic residues. They have worked well in most cases. However, the charges sometimes can have unintended effects and can interact with some molecules nonspecifically.

A new series of generic self-assembling, non-ionic peptides (Table 1) has been designed. The rationale for the design is as follows: i) selection of amino acids valine, isoleucine, phenyalanine and threonine as the self-assembling motifs. It is well known from numerous previous experiments that β-sheet structure facilitate self-assembly and the hydrophobic residues are key for self-assembly. Peptides with alternating hydrophobic and hydrophilic residues generally have high tendency to form β-sheet structures [28, 35, 56, 60-61]. The more hydrophobic the amino acids are, (e.g. with the increasing hydrophobicity, glycine, alanine, valine, isoleucine/leucine, phenylalanine) the more readily they undergo self-assembly and with better mechanical properties [31-33]. In order to form nanofiber scaffold hydrogel, the hydrophobicity plays major role. For example, when alanine is the hydrophobic residue, minimal 16-residue peptide is required [28-29]; when leucine is used, a 12-residue peptide is needed [34]; when phenylalanine is used, only 8-residue peptide is sufficient [33]. ii) Use polar, non-ionic residues instead of charged residues. It is known the β-branched amino acids have higher tendency to form β-sheet. There are only 3 β-branched amino acids, valine, isoleucine and threonine among the 20 natural amino acids. Threonine can form 3 hydrogen bonds on the —OH group (2 hydrogen acceptors and 1 donor) on the side chain thus make it soluble in water. A peptide with alternating threonine residues will form a hydrogel. These self-assembling motifs in Table 1 will form well-ordered nanofiber scaffold hydrogels. The lengths of peptides vary. It is also known the longer the peptides, the more readily they undergo self-assembly in water and culture media. The alternating sequences of TA, TV, TI, TL may behave like tissue inert PEGs.

These self-assembling peptides in Tables 1 and 2 will have very interesting structural and mechanical properties. Since these self-assembling motifs do not have charges, they will less likely to interact with others nonspecifically. In an analogy, these peptides may be similar to oligo ethylene glycol.

Peptides with identical active motifs but various mechanical and rheological properties, namely, gel strength and modulus. This can be achieved by varying the hydrophobic residues, alanine (least hydrophobic), valine (moderate hydrophobic) and isoleucine and phenylalanine (more hydrophobic).

There are numerous biologically active motifs that have been found and well studied [1-10] and more will be discovered over time. Some of these motifs are from extracellular matrices including laminin, fibronectin, collagens and others are from part of the soluble growth factors including bone marrow-homing motifs. These active motifs are directly extended from the self-assembling domain and some have been used in our previous studies with the charged self-assembling motifs [42-45, 58-59]. These designer peptides with active motifs are listed in Table 4. They have different attributes that will likely influence neural stem cell behaviors in 3D scaffold culture systems. We will: a) characterize the designer self-assembling peptides using circular dichroism (CD), dynamic light scattering (DLS), atomic force microscope (AFM), scanning electronic microscope (SEM), and b) study their mechanical properties of the designer scaffold hydrogels using a variety of instruments including micro-rheology to probe local variations and macro-rheology to probe bulk property.

Since we have carried out 3D peptide scaffold experiments using mouse neural stem cells [42-43], we will first use these cells as a model to carry out the initial tests to quickly establish the system.

We will also use commercially available human neural cells (Lonza product number PT-2599) and NIH registered human stem cells BG01V/hoG (variant hESC hOct4-GFP reporter cells) from Invitrogen (catalog number SKU#R7799-105). The BG01V/hOG cells are derived from the BG01V human embryonic stem cell (hESC) line (ATCC SCRC-2002). We will follow the established protocols and guidelines, perhaps with specific modifications as required. We will additionally test: a) the generic scaffolds listed in Tables 1, 2 and 4 plus various soluble growth factors; b) various designer biologically active peptide scaffolds without or with minimal soluble factors; c) combination of various designer biologically active peptide scaffolds with soluble growth factors. We will use a wide range of commercially available reagents, markers and specific monoclonal antibodies to comprehensively examine the human neural complex systems from the controlled differentiated system since we can control every single ingredient in the 3D scaffold cultures. We will ask: 1) What is the % of different neurons, glial cells, and other cell types? This can be exquisitely examined using a variety of specific monoclonal antibodies and other markers. 2) What are the organizations of the tissues and the locations of various cell types? 3) Can the different cell types undergo self-organization and reorganization? 4) Do the neural cells in the tissue de-differentiate into other phenotypes? 5) Can the neurons in the tissue form active synapses? We have already carried out this kind of research in 2000 [30]. 6) How do these different cell types interact and influence to each other?

After developing the human neural complex tissue model, we will carry out drug screens of Huntington disease inhibitors. The successful approach can be readily extend to other neurological diseases, including Alzheimer's and Parkinson's diseases as well as prion disease. The experiments will be carried out as follows. a) We will transfect specific construct of poly-glutamine (polyQ) of various lengths, from 25Q (control) to 103Q (Hungtington phenotype). These constructs are a gift from Susan Lindquist at Whitehead Institute/MIT. We will transfect the human neural cells with the constructs and the cells will express the exon-1 of the Huntington gene with a normal (Htt25Q-control) and expanded (Htt103Q) polyQ repeats. Our constructs also contain the proline rich region of the native Huntingtin protein, which plays a significant role in the stability of the protein, and the GFP for visualization of the aggregates; b) After successful culture, we will select the culture >90% pure cell line with the Htt25Q and Htt103Q using FACS sorting since the cells carry GFP; c) After select and continue culture the pure cell lines, we will subject these inducible cell line for high content drug screenings that specifically inhibit Htt103Q aggregation.

A lentivirus transfection system will be used for the transduction of the human neural progenitor cells. The lentiviral constructs are commercially available through Invitrogen (ViraPower Lentiviral Expression systems, T-Rex lentivirus transduction system) and the transfection protocols have been tested efficiently in many cell lines. Tissue cultures of human neural cells expressing the normal 25Q (control) and the expanded 103Q polyQ repeats (pathogenic) of the Htt protein will be performed in 96 and 384 multi well plates that can be used for statistically significant analyses and multiple drug screenings. For the evaluation of the effect of the different drugs on the viability of the neural cells we will employ the standard LIVE/DEAD assay as well as the MTT assay and tests. Confocal microscopy approaches along with the conventional fluorescence light microscope sample examination will be used to locate the Huntingtin fragments aggregates in differentiated cells and quantify these results. The critical protein concentration that shows neuron toxicity will be determined and methods to block the neuron toxicity will be investigated.

Drugs, small chemical compounds, and peptide libraries will be screened for their efficiency to minimize the toxicity effects of Htt103Q on neural cells. Many libraries are maintained by non-profit organizations and others can be obtained with fees. Different compound concentrations (from micromolar to nanomolar) will be used to test their activity to block Huntingtin toxicity and enhance neuron survival rates. In some cases, a combinatorial cocktail of these libraries can be used to investigate synergistic effects.

Large scale monitoring of the cell behavior in the 3D neural tissue culture system in the 384-well plates is feasible and therefore, an automated plate reader or another fast screening methodology of the cell viability such as fluorescence-activated cell sorting (FACS) may be employed.

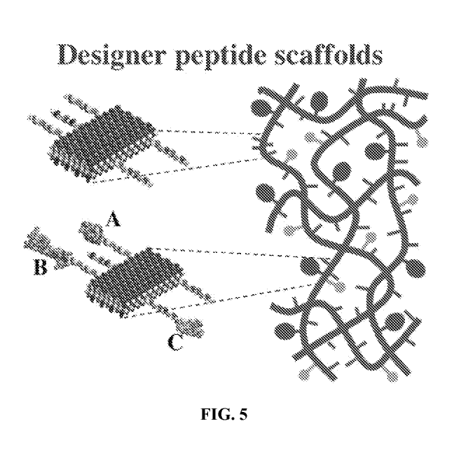

Designer peptide scaffolds so far used in diverse cell and tissue systems from a variety of sources demonstrated a promising prospect in further improvement for specific needs since tissues are known to reside in different microenvironments. The designer peptide scaffolds used thus far are general peptide nanofiber scaffolds and not tailor-made for specific tissue environment. We will produce designer peptide scaffolds to show that these designer peptide scaffolds incorporating specific functional motifs will perform as superior scaffolds in various well-controlled experiments. They may not only create a fine-tuned microenvironment for 3-D tissue cell cultures, but also may enhance cell-materials interactions, cell proliferation, migration, differentiation and performing their biological function. We can produce a fine-tuned scaffold from incorporate multiple active motifs, similar as other extracellular matrices, where several proteins co-exist in the microenvironment. This idea is depicted in FIG. 5. The designer scaffold can combine many different individual self-assembling peptides listed in Tables 1, 2 and 4.

### REFERENCES

1. Lodish, H., Berk, A., Kaiser, C. A, Krieger, M. Molecular Cell Biology, 6^{th} Edition WH Freeman, 2007.
2. Alberts B, Johnson A, Lewis J, Raff M, Roberts K, Walter P. Molecular Biology of the Cell 5th Edition, Garland Publishing, New York, 2007.
3. Ruoslahti E, Pierschbacher M D. New perspectives in cell adhesion: RGD and integrins. Science 1987; 238: 491.
4. Ruoslahti E. Fibronectin and its receptors. Annu Rev Biochem. 1988; 57: 375.
5. Yamada K M. Adhesive recognition sequences. J. Biol. Chem. 1991; 266: 12809.
6. Ratner B, Hoffman A, Schoen F, Lemons J. (Eds.) Biomaterials Science. Adademic Press, New York. 1996.
7. Lanza R, Langer R, Vacanti J. Principles of Tissue Engineering, 2^{nd} Academic Press, San Diego, Calif. USA. 2000.
8. Ayad S, Boot-Handford R P, Humphreise M J, Kadler K E, Shuttleworth C A. The extracellular matrix: Facts Book. 2^{nd}, Academic Press, 1998.
9. Kreis T, Vale R. Guide book to the extracellular matrix, anchor, and adhesion proteins. 2^{nd}. Oxford University Press. Oxford, UK, 1999.
10. Timpl et al. Laminin-a glycoprotein from basement membranes. J. Biol. Chem. 1979; 254: 9933.
11. Kleinman et al. Basement membrane complexes with biological activity. Biochemistry 1986; 25: 312.
12. Lee et al. Interaction of mouse mammary epithelial cells with collagen substrata: regulation of casein gene expression and secretion. PNAS 1985; 82: 1419.
13. Oliver et al Culture of parotid acinar cells on a reconstituted basement membrane substratum. J Dental Res 1987; 66: 594.
14. Kubota et al. Role of laminin and basement membrane in the differentiation of human endothelial cells into capillary-like structure. J Cell Biol 1988; 107: 1589.
15. Bissell M J. The differentiated state of normal and malignant cells or how to define a “normal” cell in culture. Int. Rev. Cytol. 1981; 70: 27.
16. Bissell et al., The organizing principle: microenvironmental influences in the normal and malignant breast. Differentiation 2002; 70: 537.
17. Schmeichel & Bissell, Modeling tissue-specific signaling and organ function in three dimensions. J Cell Sci. 2003; 116: 2377.
18. Weaver et al., The development of a functionally relevant cell culture model of progressive human breast cancer, Sem. Cancer Biol 1995; 6: 175.
19. Zhau, et al., Establishment of a three-dimensional human prostate organoid coculture under microgravity-simulated conditions: evaluation of androgen-induced growth and PSA expression. In Vitro Cell Dev Biol Anim, 1997; 33: 375.
20. Cukierman E, Pankov R, Stevens D R, Yamada K M., Taking cell-matrix adhesions to the third dimension. Science 2001; 294: 1708.
21. Cukierman E, Pankov R, Yamada K M., Cell interactions with three-dimensional matrices. Curr Opin Cell Biol, 2002; 14: 633-639.
22. Edelman D B, Keefer E W, A cultural renaissance: in vitro cell biology embraces three-dimensional context. Exp Neurol, 2005; 192: 1.
23. Justice B A, Badr N A, Felder R A., 3D cell culture opens new dimensions in cell-based assays. Drug Discov Today. 2008 Dec. 19. PMID:19049902.
24. Lee J, Cuddihy M J, Kotov N A., Three-dimensional cell culture matrices: state of the art. Tissue Eng Part B Rev. 2008; 14:61.
25. Seidlits S K, Lee J Y, Schmidt C E., Nanostructured scaffolds for neural applications. Nanomed., 2008; 3:183.
26. Ulijn R V, Smith A M. Designing peptide based nanomaterials. Chem Soc Rev. 2008; 37:664.
27. Zhang et al., Zuotin, a putative Z-DNA binding protein in Saccharomyces cerevisiae. EMBO. J, 1992; 11:3787.
28. Zhang et al., Spontaneous assembly of a self-complementary oligopeptide to form a stable macroscopic membrane. PNAS 1993; 90:3334.
29. Zhang et al., Self-complementary oligopeptide matrices support mammalian cell attachment, Biomaterials 1995; 16: 1385.
30. Holmes et al., Extensive neurite outgrowth and active neuronal synapses on peptide scaffolds, Proc. Natl. Acad. Sci. USA 2000; 97: 6728.
31. Caplan et al., Self-assembly of a beta-sheet oligopeptide is governed by electrostatic repulsion, Biomacromolecules 2000; 1: 627.
32. Caplan et al., Control of self-assembling oligopeptide matrix formation through systematic variation of amino acid sequence, Biomaterials, 2002; 23: 219.
33. Marini et al., Left-handed helical ribbon intermediates in the self-assembly of a beta-sheet peptide, NanoLetters 2002; 2: 295.
34. Kisiday et al., Self-assembling peptide hydrogel fosters chondrocyte extracellular matrix production and cell division: implications for cartilage tissue repair, Proc. Natl. Acad. Sci., USA 2002; 99: 9996.
35. Zhang S., Emerging biological materials through molecular self-assembly Biotechnology Advances, 2002; 20: 321.
36. Bokhari et al., The enhancement of osteoblast growth and differentiation in vitro on a peptide hydrogel-polyHIPE polymer hybrid material, Biomaterials, 2005; 26: 5198.
37. Yokoi, H., Kinoshita, T. & Zhang, S., (2005) Dynamic reassembly of peptide RADA16 nanofiber scaffold, Proc. Natl. Acad. Sci. USA, 102, 8414.
38. Chau et al., Incorporation of a matrix metalloproteinase-sensitive substrate into self-assembling peptides—a model for biofunctional scaffolds, Biomaterials 2008; 29, 1713.
39. Zhang et al., Design nano biological materials through self-assembly of peptide & proteins, Current opinion in Chemical Biology, 2002; 6: 865.
40. Zhang S., Fabrication of novel materials through molecular self-assembly, Nature Biotechnology 2003; 21: 1171.
41. Zhang S., Beyond the Petri Dish, Nature Biotechnology, 2004; 22, 151.
42. Gelain et al., Systematic studies of self-assembling peptide nanofiber scaffold with other scaffolds, J. Nanoscience & Nanotechnology, 2007; 11, 424.
43. Gelain et al., Designer self-assembling peptide nanofiber scaffolds for 3-D culture of adult mouse neural stem cells, PLoS ONE 2006; 1, e119.
44. Horii et al., Biological designer self-assembling peptide scaffolds significantly enhance osteoblast proliferation, differentiation and 3-D migration, PLoS ONE 2007; 2, e190.
45. Wang, X., Horii, A. & Zhang, S., Designer functionalized self-assembling peptide nanofiber scaffolds for growth, migration, and tubulogenesis of human umbilical vein endothelial cell, Soft Matter 2008; 4, 2388.
46. Nagai et al., Diffusion coefficients in self-assembling peptide nanofiber scaffold hydrogel, J. Controlled Release, 2006; 115, 18.
47. Koutsopoulos et al., Slow release of functional proteins through designer self-assembling peptide nanofiber hydrogel scaffolds, PNAS 2009; (in press).
48. Ellis-Behnke et al., Nano neuro knitting: peptide nanofiber scaffold for brain repair and axon regeneration with functional return of vision, PNAS 2006; 103, 5054.
49. Guo et al., Reknitting the injured spinal cord by self-assembling peptide nanofiber scaffold. Nanomedicine, 2007; 3:311.
50. Ellis-Behnke et al., Nano hemostat solution: immediate hemostasis at the nanoscale. Nanomedicine, 2007; 2, 207.
51. Davis et al., Injectable self-assembling peptide nanofibers create intramyocardial microenvironments for endothelial cells, Circulation 2005; 111: 442.
52. Davis et al., Local myocardial IGF-1 delivery with biotinylated peptide nanofibers improves cell therapy for myocardial infarction, PNAS 2006; 103, 8155.
53. Narmoneva et al., Self-assembling short oligopeptides and the promotion of angiogenesis, Biomaterials, 2005; 26: 4837.
54. Zhang et al., Designer self-assembling peptide nanofiber scaffolds for 3-D tissue cell cultures, Seminars in Cancer Biology, 2005; 15, 413.
55. Zhang et al., PuraMatrix: Self-assembling peptide nanofiber scaffolds, In Scaffolding in Tissue Engineering, 2005; CRC Press, Boca Raton, Fla. p 217.
56. Zhang et al., Molecular Design of Biological and Nano-Materials in Biomimetics: Mimicking and Inspiration of Biology, 2005; CRC Press pp. 229.
57. Zhang, S., Designing novel materials and molecular machines, In The Promise of Biotechnology US State Department e-Journal, 2005; 10: 22.
58. Yang, Y. Khoe, U., Wang, X., Horii, A., Yokoi, H., & Zhang, S., Designer self-assembling peptides nanomaterials, Nano Today 2009; (in press).
59. Gelain et al. Designer self-assembling peptide scaffolds for 3D tissue cell cultures. Method in Bioengineering, Artech House. 2009, (in press).
60. Branden, C, & Tooze J., Introduction to Protein Structure, 2^{nd} Ed. Garland Publishing, New York, N.Y. 1999.
61. Petsko G A, Ringe D. Protein structure and function, New Science Press Ltd. London, UK. 2003.
62. Koutsopoulos, S., Lindquist, S. L. & Zhang, S., A 3D complex tissue model to study Huntington's disease using neural stem cells (in preparation).

While this invention has been particularly shown and described with references to preferred embodiments thereof, it will be understood by those skilled in the art that various changes in form and details may be made therein without departing from the scope of the invention encompassed by the appended claims.

What is claimed is:

1. An isolated peptide comprising a self-assembling motif, wherein the self-assembling motif has an amino acid sequence having a formula selected from the group consisting of:
[(τ)ₖ(Φ)ₚ]ₘ(τ)ₙ (Formula I); and
[(Φ)ₚ(τ)ₖ]ₘ(Φ)ₙ (Formula II);
wherein each (τ) is independently a non-ionic, polar amino acid;
wherein each (Φ) is independently a hydrophobic amino acid;
wherein k is an integer greater than or equal to 1;
wherein p is an integer greater than or equal to 1;
wherein m is an integer greater than or equal to 3;
wherein n is 0 or 1; and
wherein each amino acid in the peptide is a D-amino acid or each amino acid is a L-amino acid.

2. The peptide of claim 1, wherein k is 1.

3. The peptide of claim 1, wherein p is 1.

4. The peptide of claim 1, wherein all amino acids in the self-assembling motif are natural amino acids.

5. The peptide of claim 4, wherein each (Φ) is a hydrophobic amino acid independently selected from the group consisting of alanine, valine, isoleucine leucine and phenylalanine.

6. The peptide of claim 4, wherein each (τ) is independently selected from the group consisting of serine or threonine, tyrosine and hydroxy-proline.

7. The peptide of claim 5, wherein each (τ) is independently selected from the group consisting of serine or threonine, tyrosine and hydroxy-proline.

8. The peptide of claim 1, wherein each (τ) is the same non-ionic, polar amino acid.

9. The peptide of claim 1, wherein (τ) is serine, threonine, tyrosine or hydroxy-proline.

10. The peptide of claim 9, wherein (τ) is threonine.

11. The peptide of claim 1, wherein each (Φ) is the same hydrophobic amino acid.

12. The peptide of claim 11, wherein (Φ) is selected from the group consisting of alanine, valine, isoleucine, leucine and phenylalanine.

13. The peptide of claim 8, wherein each (Φ) is the same hydrophobic amino acid.

14. The peptide of claim 13, wherein (Φ) is selected from the group consisting of alanine, valine, isoleucine, leucine and phenylalanine.

15. The peptide of claim 14, wherein (τ) is threonine.

16. The peptide of claim 1, wherein the C-terminus of the self-assembling motif is amidated.

17. The peptide of claim 1, wherein the N-terminus is acetylated.

18. The peptide of claim 17, wherein the C-terminus of the self-assembling motif is amidated.

19. The peptide of claim 15, wherein the self-assembling motif is selected from the group consisting of:

| | |
|---|---|
| ATATATATATATATATA; | (SEQ ID NO: 1) |
| VTVTVTVTVTVTVT; | (SEQ ID NO: 2) |
| VTVTVTVTVTVT; | (SEQ ID NO: 3) |
| TITITITITITIT; | (SEQ ID NO: 4) |
| TITITITITIT | (SEQ ID NO: 5) |
| TFTFTFTFTFT; | (SEQ ID NO: 6) |
| and | |
| TFTFTFTFT. | (SEQ ID NO: 7) |

20. The peptide of claim 15, wherein the self-assembling motif is selected from the group consisting of:

| | |
|---|---|
| Ac-ATATATATATATATATA-NH₂; | (SEQ ID NO: 8) |
| Ac-VTVTVTVTVTVTVT-NH₂; | (SEQ ID NO: 9) |
| Ac-VTVTVTVTVTVT-NH₂; | (SEQ ID NO: 10) |
| Ac-TITITITITITIT-NH₂; | (SEQ ID NO: 11) |
| Ac-TITITITITIT-NH₂; | (SEQ ID NO: 12) |
| Ac-TFTFTFTFTFT-NH₂; | (SEQ ID NO: 13) |
| and | |
| Ac-TFTFTFTFT-NH₂. | (SEQ ID NO: 14) |

21. The peptide of claim 1, wherein the peptide further comprises a biologically active motif.

22. The peptide of claim 21, wherein the peptide further comprises a glycine linker.

23. The peptide of claim 21, wherein the biologically active motif is from an extracellular motif.

24. The peptide of claim 23, wherein the extracellular motif is selected from the group consisting of laminin, fibronectin and collagen.

25. The peptide of claim 21, wherein the biologically active motif has an amino acid sequence selected from the following table:

| SEQ ID NO | Peptide sequence |
|---|---|
| 15 | AASIKVAVSADR |
| 16 | CSRARKQAASIKVAVSADR |
| 17 | YIGSR |
| 18 | PDGSR |
| 19 | RYVVLPR |
| 20 | KAFDITYVRLKF |
| 21 | TAGSCLRKFSTM |
| 22 | RNIAEIIKDI |
| 23 | YVRL |
| 24 | IRVTLN |
| 25 | TTVKYIFR |
| 26 | SIKIRGTY |
| 27 | RQVFQVAYIIIKA |
| 28 | FQIAYVIVKA |
| 29 | GQLFHVAYIIIKA |
| 30 | FHVAYVLIKA |
| 31 | LENGEIVSLVNGR |
| 32 | LGTIPG |
| 33 | DGEA |
| 34 | REDV |
| 35 | GVGVP |
| 36 | GVGVAP |
| 37 | IKVAV |
| 38 | PFSSTKT |
| 39 | SKPPGTSS |
| 40 | SDPGYIGSR |
| 41 | RNIAELLKDI |
| 42 | PRGDSGYRGDSG |
| 43 | GFLGFPT |
| 44 | YGPDSGR. |

26. The peptide of claim 21 having an amino acid selected from the following table:

| SEQ ID | |
|---|---|
| NO | Sequence |
| 45 | Ac-ATATATATATATATATAYGPDSGR-NH2 |
| 46 | Ac-ATATATATATATATATAGIKVAV-NH2 |
| 47 | Ac-ATATATATATATATATAGYIGSR-NH2 |
| 48 | Ac-ATATATATATATATATAGPFSSTKT-NH2 |
| 49 | Ac-ATATATATATATATATAGSKPPGTSS-NH2 |
| 50 | Ac-ATATATATATATATATAGGSDPGYIGSR-NH2 |
| 51 | Ac-ATATATATATATATATAGGRNIAELLKDI-NH2 |
| 52 | Ac-ATATATATATATATATAGPRGDSDGYRGDSG-NH2 |
| 53 | Ac-VTVTVTVTVTVTVTGYGPDSGR-NH2 |
| 54 | Ac-VTVTVTVTVTVTVTGIKVAV-NH2 |
| 55 | Ac-VTVTVTVTVTVTVTGYIGSR-NH2 |
| 56 | Ac-VTVTVTVTVTVTVTGPFSSTKT-NH2 |
| 57 | Ac-VTVTVTVTVTVTVTGSKPPGTSS-NH2 |
| 58 | Ac-VTVTVTVTVTVTVTGGSDPGYIGSR-NH2 |
| 59 | Ac-VTVTVTVTVTVTVTGGRNIAELLKDI-NH2 |
| 60 | Ac-VTVTVTVTVTVTVTGPRGDSGYRGDSG-NH2 |
| 61 | Ac-TITITITITITITGYGPDSGR-NH2 |
| 62 | Ac-TITITITITITITGIKVAV-NH2 |
| 63 | Ac-TITITITITITITGYIGSR-NH2 |
| 64 | Ac-TITITITITITITGPFSSTKT-NH2 |
| 65 | Ac-TITITITITITITGSKPPGTSS-NH2 |
| 66 | Ac-TITITITITITITGGSDPGYIGSR-NH2 |
| 67 | Ac-TITITITITITITGGRNIAELLKDI-NH2 |
| 68 | Ac-TITITITITITITGPRGDSGYRGDSG-NH2 |
| 69 | Ac-TFTFTFTFTFTGYGPDSGR-NH2 |
| 70 | Ac-TFTFTFTFTFTGIKVAV-NH2 |
| 71 | Ac-TFTFTFTFTFTGYIGSR-NH2 |
| 72 | Ac-TFTFTFTFTFTGPFSSTKT-NH2 |
| 73 | Ac-TFTFTFTFTFTGSKPPGTSS-NH2 |
| 74 | Ac-TFTFTFTFTFTGGSDPGYIGSR-NH2 |
| 75 | Ac-TFTFTFTFTFTGGRNIAELLKDI-NH2 |
| 76 | Ac-TFTFTFTFTFTPRGDSGYRGDSG-NH2. |

27. A self-assembled nanostructure comprising a peptide of claim 1.

28. An aqueous mixture comprising a plurality of peptides of claim 1, wherein said peptides are capable of self-assembly.

29. The composition of claim 28, wherein the mixture is a heterogenous mixture.

30. The composition of claim 28, wherein the mixture is a homogenous mixture.

31. A method of preparing a self-assembled nanostructure comprising forming an aqueous mixture of peptides under conditions suitable for self-assembly of the peptides, wherein each peptide has a formula selected from:
[(τ)ₖ(Φ)ₚ]ₘ(τ)ₙ (Formula I); and
[(Φ)ₚ(τ)ₖ]ₘ(Φ)ₙ (Formula II);
wherein each (τ) is independently a non-ionic, polar amino acid;
wherein each (Φ) is independently a hydrophobic amino acid;
wherein k is an integer greater than or equal to 1;
wherein p is an integer greater than or equal to 1;
wherein m is an integer greater than equal to 3;
wherein n is 0 or 1;
wherein each amino acid in the peptide is a D-amino acid or each amino acid is a L-amino acid; and
wherein said peptides are capable of self-assembly.

32. The method of claim 31, wherein the aqueous mixture further comprises a salt.

33. An isolated peptide wherein said peptide comprises an amino acid sequence having a formula selected from:
[(τ)ₖ(Φ)ₚ]ₘ(τ)ₙ (Formula I); and
[(Φ)ₚ(τ)ₖ]ₘ(Φ)ₙ (Formula II);
wherein each (τ) is independently a non-ionic, polar amino acid;
wherein each (Φ) is independently a hydrophobic amino acid;
where k is an integer greater than equal to 1;
wherein p is an integer greater than equal to 1;
wherein m is an integer greater than equal to 3;
wherein n is 0 or 1;
wherein each amino acid in the peptide is a D-amino acid or each amino acid is a L-amino acid.

34. The peptide of claim 33, wherein each (τ) is independently threonine or serine; and wherein each (Φ) is independently a hydrophobic amino acid selected from the group consisting of alanine, valine, isoleucine, leucine, phenylalanine.

35. A macroscopic material which is formed by self-assembly of self-assembling peptides in an aqueous solution;
wherein said peptides comprise a self-assembling motif having an amino acid sequence selected from the group consisting of:
[(τ)ₖ(Φ)ₚ]ₘ(τ)ₙ (Formula I); and
[(Φ)ₚ(τ)ₖ]ₘ(Φ)ₙ (Formula II);
wherein each (τ) is independently a non-ionic, polar amino acid;
wherein each (Φ) is independently a hydrophobic amino acid;
wherein k is an integer greater than or equal to 1;
wherein p is an integer greater than or equal to 1;
wherein m is an integer greater than equal to 3; and
wherein n is 0 or 1;
wherein each amino acid in the peptide is a D-amino acid or each amino acid is a L-amino acid.

36. The material of claim 35, wherein said material is composed of β-sheets.

37. A macroscopic scaffold comprising a plurality self-assembling peptides,
wherein said peptides comprise a self-assembling motif having an amino acid sequence selected from the group consisting of:
[(τ)ₖ(Φ)ₚ]ₘ(τ)ₙ (Formula I); and
[(Φ)ₗ(τ)ₚ]ₘ(Φ)ₙ (Formula II);
wherein each (τ) is independently a non-ionic, polar amino acid;
wherein each (Φ) is independently a hydrophobic amino acid;
wherein k is an integer greater than or equal to 1;
wherein p is an integer greater than or equal to 1;
wherein m is an integer greater than or equal to 3;
wherein n is 0 or 1;
wherein each amino acid in the peptide is a D-amino acid or each amino acid is a L-amino acid;
wherein said peptides self-assemble into a β-sheet macroscopic scaffold; and wherein said macroscopic scaffold encapsulates living cells, said cells being present in said macroscopic scaffold in a three-dimensional arrangement.

38. A method of regenerating a tissue, said method comprising administering to a mammal a solution comprising self-assembling peptides and living cells;
wherein said peptides comprise a self-assembling motif having an amino acid sequence selected from the group consisting of:
[(τ)ₖ(Φ)ₚ]ₘ(τ)ₙ (Formula I); and
[(Φ)ₚ(τ)ₖ]ₘ(Φ)ₙ (Formula II);
wherein each (θ) is independently a non-ionic, polar amino acid;
wherein each (Φ) is independently a hydrophobic amino acid;
wherein k is an integer greater than or equal to 1;
wherein p is an integer greater than or equal to 1;
wherein m is an integer greater than or equal to 3;
wherein n is 0 or 1;
wherein said peptides do not substantially self-assemble prior to said administration; and
wherein said peptides self-assemble into a β-sheet macroscopic scaffold after said administration, thereby encapsulating said cells in vivo, said cells being present in said macroscopic scaffold in a three-dimensional arrangement.

39. A method for in vitro cell culture comprising:
a) adding a macroscopic material of claim 35, to a cell culture medium comprising cells, thereby forming a membrane/culture mixture; and
b) maintaining the mixture under conditions sufficient for cell growth.
